(19) United States
(12) Patent Application Publication
(10) Pub. No.: US 2004/0029953 A1
(43) Pub. Date: Feb. 12, 2004

(54) Use of neuropeptide-y antagonists in treatment of alcoholism

(76) Inventor: Clyde W. Hodge, Chapel Hill, NC (US)

Correspondence Address: Jonathan Alan Quine, Quine Intellectual Property Law Group, P O Box 458, Alameda, CA, 94501, US

(21) Appl. No.: 10/398,091

(22) PCT Filed: Oct. 2, 2001
(86) PCT No.: PCT/US01/30830

(30) Foreign Application Priority Data
Oct. 3, 2000 (US) 60237964

(51) Int. Cl. A61K 31/353

(52) U.S. Cl. 514/455

(57) ABSTRACT

The present invention provides a method of treating of alcoholism and alcohol abuse in a mammal comprising administering a therapeutically effective amount of an NPY receptor antagonist. The present invention is also directed to pharmaceutical compositions containing the same.

### ACKNOWLEDGEMENTS

This invention was supported in part by grant #AA1 1854 from the National Institutes of Health. The U.S. Government may have rights in this invention.

### INTRODUCTION

1. Field of Invention

The invention relates to compositions and methods for the treatment of alcoholism and alcohol abuse in mammals.

2. Background

Alcohol abuse is one of the most significant problems in modern society. According to the National Institutes of Health, each year alcohol abuse accounts for 45% of all car crash fatalities (over 20,000 individuals) and is involved in approximately 44% of all short-stay hospital visits. An additional 25,000 individuals die from alcohol-associated cirrhosis of the liver (NIH Publication No. 97-4017, 1997). The Justice Department reported that alcohol was involved in nearly 40% of all violent crimes in 1998. The resulting economic cost of alcohol abuse to the United States is estimated to be nearly $150 billion per year.

Disulfiram (Antabuse®) and Naltrexone (Trexan®) are the only FDA approved products that are currently available for adjunctive use in the treatment of alcohol abuse; Disulfiram works by blocking the intermediary metabolism of alcohol in the body to produce a build up of acetaldehyde, which in turn produces markedly adverse behavioral and physiological effects. Patient compliance in taking the drug is poor due to these side effects. (see T. W. Rall, in: Goodman and Gilman's The Pharmacological Basis of Therapeutics, A. G. Gilman et al., 8^{th} Edition, Chap. 17, pp. 378-379). Naltrexone is a well-known narcotic antagonist and is thought to work by blocking activation of the endogenous opiate reward system, which may be activated by alcohol consumption. In practice, naltrexone is only moderately effective because it is relatively short acting and patients require co-treatment with behavioral therapy for the drug to have any effect (J. R. Volpicelli et al., Arch. Gen. Psychiatry, 1992, 49:876-880). Thus, it is of interest to develop novel methods and compositions that are useful for the treatment of for the treatment of alcoholism and alcohol abuse in mammals.

The neurobiological mechanisms by which alcohol interacts with brain and behavioral processes to produce addiction are not fully characterized. Research on other excessive consummatory behaviors, such as obesity, has identified the hypothalamus as a primary central nervous system regulatory system (see J. E. Blundell, Appetite, 1986, 7:3956; S. P. Kalra et al., Endocr. Rev., 1999, 20:68,100). Recent evidence indicates that alcohol and food intake are similarly regulated by hypothalamic transmitter systems. Injections of serotonin (5-HT) in the paraventricular nucleus (PVN) inhibit norepinephrine-induced carbohydrate intake (S. F. Leibowitz and G. Shor-Posner, Appetite, 1986, 7:Suppl 1-14.). Similarly, site-specific norepinephrine infusion in the PVN also produces increases in alcohol self-administration, which are completely blocked by co-infusion of 5-HT (C. W. Hodge et al., Alcohol Clin. Exp. Res.,1996, 20:1669-1674.). This result suggests that feeding and alcohol-seeking behavior may be regulated by common hypothalamic mechanisms (H. H. Samson and C. W. Hodge, “Neurobehavioral regulation of ethanol intake,” in Pharmacological Effects of Ethanol on the Nervous System, R. A. Deitrich and V. G. Erwin, eds., pp 203-226, CRC Press, Boca Raton, 1996).

Neuropeptide Y (NPY), a 36-amino-acid residue peptide, is the most potent stimulant of feeding behavior known. Infusions of NPY into the cerebral ventricles or into nuclei of the hypothalamus (B. G. Stanley et al., Peptides, 1995, 6:1205-¹²¹I; B. G. Stanley et al. Brain Res, 1993, 604:304-317) increase food intake (B. G. Stanley and S. F. Leibowitz, Life Sci., 1994, 35:2635-2642), and repeated injections lead to hyperphagia and obesity (B. G. Stanley et al., Peptides, 1996, 7:1189-1192). Injection of NPY into a number of brain regions leads to increased food intake, but the PVN is the primary site of action (B. G. Stanley et al., Proc. Natl. Acad. Sci. USA, 1985, 82:3940-3943; Stanley et al., 1993). The NPY receptor is known to exist in various subtypes, which respond to subtype-selective antagonists (A. Balasubramanian, Peptides, 1997, 18:445-457). Considerable attention has been paid to the receptor subtype mediating the food craving or orexigenic effect of NPY (C. Gerald et al., Nature, 1996, 382:168-171; D. O'Shea et al., Endocrinology, 1997, 138:196-202; Y. H. Hu et al., J. Biol. Chem., 1996, 271:26315-26319). The Y1 receptor was originally proposed as the receptor involved in this effect, since the Y1 agonist [Pro]NPY stimulates feeding. However, potent orexigenic effects are also produced by an N-terminal truncated NPY fragment, NPY 2-36, which has low potency at the Y1 receptor, which led to the concept that a novel “Y 1-like” receptor may mediate the effect (G. Stanley et al., Peptides, 1992, 13:581-587). In addition, [Pro³⁴]-NPY stimulates approximately 50% of the food intake seen following the injection of NPY, suggesting that the Y1 receptor may be responsible for some portion of the orexigenic effect of NPY (O'Shea et al., 1997).

Non-peptide NPY Y1 receptor-selective antagonists are known. H. N. Doods et al. reported the design, selectivity and cardiovascular properties of Y1-selective BIBP 3226 (Regul. Pept., 1996, 65:71-77). U.S. Pat. No. 5,616,620 discloses BIBP 3226 and its analogs as useful in treatment of cardiovascular diseases, obesity and diabetes. BIBO 3304 is a non-peptide antagonist with subnanomolar affinity for the Y1 receptor subtype that significantly inhibits food intake in rats induced by application of NPY or by fasting (H. A. Wieland et al., Br. J. Pharmacol.,1998, 125:549-55). U.S. Pat. No. 6,114,390 discloses BIBO 3304 and its analogs as useful in treatment of numerous diseases and disorders including hypertension, cardiovascular diseases, obesity and diabetes. Non-peptide NPY Y5 receptor-selective antagonists are also known to affect feeding behavior (Kanatani et al., Biochem. Biophys. Res. Commun., 2000, 272:169-173).

Relevant Literature

Several studies have implicated NPY in the biochemical, physiological, and behavioral effects of ethanol. Selectively bred alcohol-preferring (P) rats have lower levels of NPY-like immunoreactivity in the hippocampus, amygdala, and frontal cortex as compared to alcohol-non-preferring (NP) rats (C. L. Ehlers et al., Alcohol Clin. Exp. Res., 1998a, 22:1778-1782). However, P rats have more NPY immunoreactivity in the PVN and arcuate nucleus than NP rats (B. H. Hwang et al., Alcohol Clin. Exp. Res., 1999, 23(6):1023-1030). Similarly, long-term exposure to a 6% ethanol-containing diet produced elevated NPY content in the PVN and altered feeding patterns of Long-Evans rats (J. T. Clark et al., Regul. Pept., 1998, 75-76:335-345). Genetic linkage analysis of P and NP rats identified a significant quantitative trait locus on a chromosomal region that includes the NPY gene (L. G. Carr et al., Alcohol Clin. Exp. Res., 1998, 22:884-887). Comparisons of mutant mice that lack NPY with transgenic mice that overexpress NPY show that ethanol intake and acute sensitivity are inversely related to total levels of NPY in the brain (T. E. Thiele et al., Nature, 1998, 396:366-369).

Selectively bred high-alcohol-drinking (HAD) rats have less NPY immunoreactivity in the PVN than low-alcohol-drinking (LAD) rats (Hwang et al., 1999). In addition, null mutant mice lacking NPY drink more ethanol than wild-type control mice and transgenic mice that over express NPY drink less ethanol than wild-type control mice (Thiele et al., 1998). Taken together, these data suggest that reduced NPY levels are associated with increases in ethanol self-administration. However, HAD rats also have lower levels of NPY in the central amygdala as compared to LAD rats (Hwang et al., 1999) indicating that alterations in NPY levels in other brain regions might influence ethanol self-administration in these particular rats. In addition, the behavior of NPY null mutant and transgenic mice was likely influenced by global changes in NPY as well as potential developmental compensation in other functionally related peptidergic systems, which makes it difficult to draw any specific conclusions regarding the role of hypothalamic NPY in ethanol self-administration from that study.

### SUMMARY OF THE INVENTION

The present invention provides a method of treating alcoholism and alcohol abuse in a mammal comprising administering a therapeutically effective amount of an NPY receptor antagonist. The present invention is also directed to pharmaceutical compositions containing the same.

In an aspect of the present invention, activation of NPY receptors by NPY (or other ligand) binding to the receptors in the PVN is prevented or decreased by administration of an NPY receptor antagonist. In one embodiment the invention provides a method for reducing self-administration of alcohol by a patient suffering from alcoholism. In another embodiment the invention provides a method for reducing alcohol-seeking behavior in a patient suffering from alcoholism. In yet another embodiment, the invention provides method for preventing or reducing the occurrence of relapse drinking in a recovering alcoholic patient. These aspects are accomplished by the administration of a therapeutically effective amount of an NPY receptor antagonist. Depending upon the object desired, the therapeutically effective amount is sufficient to reduce alcohol self-administration and preference in the alcoholic patient, is sufficient to reduce alcohol-seeking behavior in the alcoholic patient or is sufficient to reduce the occurrence of relapse drinking of alcohol in a recovering alcoholic patient, thereby treating the alcoholism and alcohol abuse. The invention finds use in the treatment of alcoholism, alcohol dependence or alcohol abuse, for decreasing craving for alcohol, for suppressing an urge for alcohol, and for limiting alcohol consumption in an individual whether or not the individual is genetically predisposed to alcoholism or alcohol abuse.

Other objects of the invention may be apparent to one skilled in the art upon reading the following specification and claims.

### BRIEF DESCRIPTION OF THE DRAWINGS

FIG. 1 depicts the effects of NPY or NPY+D-NPY infused in the PVN immediately before 1-hour test sessions on (A) ethanol intake, (B) preference, and (C) water intake. Drug doses were administered in random order. Data are plotted as Mean±SEM of 11 rats. (*) indicates significantly different from vehicle control, Dunnett p<0.05. (t) indicates significantly different from NPY alone, paired-t-test p<0.05.

FIG. 2 depicts the effects of NPY or NPY+D-NPY infused in the PVN on (A) body weight, (B) food intake, and (C) water intake measured in the home cage 24 hours after infusion. Drug doses were administered in random order. Measurements were taken on the same days as the data shown in FIG. 1. Data are plotted as Mean±SEM of 11 rats. (*) indicates significantly different from vehicle control, Dunnett p<0.05.

FIG. 3 depicts effects of NPY, BIBP 3226, or NPY+BIBP 3226 infused in the PVN immediately before 1-hour test sessions on (A) ethanol intake, (B) preference, and (C) water intake. Drug doses were administered in random order. Data are plotted as Mean±SEM of 9 rats. (*) indicates significantly different from vehicle control, paired-t-test p<0.05.

FIG. 4 depicts a dose response curve showing the effects the NPY-Y1 antagonist BIBP 3226 in the central nucleus of the Amygdala. BIBP 3226 significantly reduced the dose of alcohol that was self-administered during 1-hour sessions. *—Indicates significantly different from vehicle (veh), Tukey test (p<0.05), N=9 rats.

FIG. 5 Total ethanol reinforced lever presses plotted as a function of time (hour) of behavioral test sessions with trained C57BL/6J mice. Administration of 60 mg/kg of L152,804 (open circles) or saline solution (closed circles) were compared. Alcohol self-administration peaked during the 4^{th} and 5^{th} hour of access. LI 52,804 blocked this peak in alcohol-seeking behavior *—Indicates significantly different from saline control at the corresponding time point.

FIG. 6—Response Latency (i.e., delay to the first alcohol lever press) plotted as a function of dose of L 152,804. L 152,804 dose dependently delayed the onset of responding. *—Indicates significantly different from no injection (ni) and saline (sal) controls, Tukey test, P<0.05. The highest dose did not achieve significance due to variability of two data points, which were almost 2 standard deviations above the mean. This indicates a very potent effect in these two mice.

### DESCRIPTION OF SPECIFIC EMBODIMENTS

The present invention provides a method of treating alcoholism and alcohol abuse in a mammal comprising administering a therapeutically effective amount of an NPY receptor antagonist. In one embodiment of the present invention, NPY receptors in the PVN are blocked by administration of a therapeutically effective amount of an NPY receptor antagonist. The therapeutically effective amount is sufficient to decrease ethanol self-administration and preference in an affected mammal, thereby treating alcohol dependence and alcohol abuse by the medical management of excessive alcohol consumption. In related aspects the invention provides a method for reducing self-administration of alcohol in a patient suffering from alcoholism comprising administering to said patient a therapeutically effective amount of an NPY receptor antagonist and determining the level of alcohol self-administration in said patient before and after said administering, a method for reducing alcohol-seeking behavior in a patient suffering from alcoholism comprising administering to said patient a therapeutically effective amount of an NPY receptor antagonist and determining the level of alcohol-seeking behavior in said patient before and after said administering, and a method for preventing or reducing the occurrence of relapse drinking in a recovering alcoholic patient comprising administering to said patient a therapeutically effective amount of an NPY receptor antagonist and determining the frequency of occurrence of relapse drinking in said patient before and after said administering. All of these aspects relate to the general, overall goal of treating alcoholism and alcohol abuse.

In a human, alcohol dependence and alcohol abuse are characterized by any of the following symptoms: (1) marked tolerance, which is the need for markedly increased amounts of alcohol (at least 50 percent increase) in order to achieve intoxication or desired effect, or markedly diminished effect with continued use of the same amount of alcohol; (2) characteristic withdrawal symptoms for alcohol; (3) alcohol frequently taken to relieve or avoid withdrawal symptoms; (4) persistent desire or one or more unsuccessful efforts to cut down or control drinking; (5) consumption of alcohol in larger amounts or over a longer period than intended; (6) important social, occupational, or recreational activities given up or reduced because of alcohol consumption; (7) large amounts of time spent in activities necessary to obtain alcohol, to drink, or to recover from its effects; (8) frequent intoxication or withdrawal symptoms when expected to fulfill major role obligations at work, school, or home; or (9) continued drinking despite knowledge of having a persistent or recurrent social, psychological, or physical problem that is caused or exacerbated by alcohol use. Typically, these symptoms persist for at least one month or have occurred repeatedly over a longer period of time. Alcohol abuse is particularly characterized by clinically significant impairment or distress, as manifested by one or more of the following occurring within a 12-month period: (1) recurrent drinking resulting in a failure to fulfill major role obligations at work, school, or home; (2) recurrent drinking in situations in which it is physically hazardous; (3) recurrent alcohol-related legal problems; or (4) continued alcohol use despite having persistent or recurrent social or interpersonal problems caused by the effects of alcohol.

In another embodiment of the invention, an amount of an NPY receptor antagonist sufficient to block the effects of NPY in an alcoholic mammalian host and to decrease craving for alcohol is administered. The invention finds particular use in preventing relapse drinking in recovering alcoholics. Elevated NPY levels in the brain correlate with dramatic increases in alcohol-seeking behavior and with intense cravings for alcohol. Blocking the effects of NPY at its receptors decreases these cravings and diminishes the likelihood of relapse drinking.

In the present invention, an “NPY receptor antagonist” or an “NPY antagonist” refers to a compound or composition that serves to block the action of endogenous or exogenous neuropeptide-Y (NPY) on NPY receptors in the brain or periphery such that alcohol self-administration is reduced. Preferably, the NPY antagonist reduces alcohol craving and self-administration of alcohol and does not adversely affect normal food or water consumption. An NPY antagonist that is non-selective is one that binds to multiple NPY receptor subtypes including the Y1 and/or the Y5 receptor subtypes. An example of a non-selective NPY antagonist that finds use in the present invention is [D-Tyr^{27,36},D-Thr³²] Neuropeptide Y (27-36), which is abbreviated as D-NPY. D-NPY, which binds with antagonistic properties to NPY Y1, Y2, Y4 and Y5 receptor subtypes, may be obtained as described by R. D. Meyers et al., in Brain Res. Bull., 1995, 37: 237-245, which is herein incorporated by reference. Another non-selective NPY antagonist that finds use in the present invention is BW1229U91, which displays a high nanomolar affinity for Y1 and Y4 receptors, a moderate affinity for Y5 receptors, but has a much lower affinity for Y2 receptors. BW1220U91 may be obtained as described by P. S. Widdowson et al, in Peptides, 1999, 20:367-372, which reference is incorporated herein by reference.

In a preferred embodiment, the NPY receptor antagonist is selective for the NPY Y1-receptor subtype. An example of a Y1-selective antagonist useful in the present invention is (R)-N²-(diphenylacetyl)-N-[(4-hydroxyphenyl)methyl]-D-arginine amide, also know as BIBP 3226. BIBP 3226 may be obtained as described in U.S. Pat. No. 5,616,620 and by H. N. Doods et al., in Regulatory Peptides, 1996, 65:71-77, both of which are herein incorporated by reference. Other useful Y1-selective antagonists include analogs of BIBP 3226 such as:

(R)-N²-[(Bis(4-bromphenyl) acetyl]-N-[(4-hydroxyphenyl)-methyl]-argininamide,

(R)-N²-(Diphenylacetyl)-N-[[4-(2-hydroxyethyl)phenyl]-methyl]argininamide,

(R)-N²Diphenylacetyl)-N-[(4-hydroxyphenyl)methyl]-argininamide,

(R)-N²-Diphenylacetyl)-N-[[4-hydroxymethyl)phenyl]-methyl]-argininamide,

N²-(Diphenylacetyl)-N-[4-hydroxy-3-methylphenyl]-methyl] argininamide,

(R,S)-3-[3-(aminoiminomethyl)phenyl]-N²-(diphenylacetyl)-N-[(4hydroxyphenyl)methyl]-alaninamide,

(R)-N²-(Diphenylacetyl-N-[(4-hydroxyphenyl)methyl]-N-methyl-argininamide,

(R,S)-N²-(Diphenylacetyl)-N-[(4-hydroxyphenyl)methyl]-N′-(1H-imidazol-2-yl)ornithinamide,

N-[(3,5-Dimethyl-4-hydroxyphenyl)methyl]-N²-(diphenylacetyl)-argininamide,

N²-(Diphenylacetyl)-N-[(4-methoxyphenyl)methyl]-argininamide, and

(R)-N-[[4-[(4,5-Dihydro-5,5-dimethyl-2,4(3H)-dioxo-1H-imidazol-3yl)methyl]phenyl]methyl]-N²-(diphenylacetyl)-argininamide,

and pharmaceutically acceptable salts and hydrates thereof.

A further example of a NPY Y1 receptor antagonist with utility in the present invention is (R)-N-[[4-(aminocarbonylaminomethyl)-phenyl]methyl]-N²-(diphenylacetyl)arginine amide trifluoroacetate, also know as BIBO 3304. B 30 3304 may be obtained according to the method described in U.S. Pat. No. 6,114,390 and by H. A. Wieland et al., in Br. J. Pharmacol., 1998, 125:549-55, both of which are herein incorporated by reference. Other useful Y1-selective antagonists include analogs of BIBO 3304 such as:

(R)-N-[[4-(aminocarbonylaminomethyl)phenyl]methyl]-N²-(diphenylacetyl)argininamide;

(R,S)-N-5-(aminoiminomethyl)-N²-(diphenylacetyl)-N-[(4-hydroxyphenyl)methyl]N⁵-methyl-ornithinamide;

(R)-N-[[4-(aminocarbonyphethyl)phenyl]methyl]-N²-(diphenylacetyl)-argininamide;

(R)-N²-(diphenylacetyl)-N-[[4-(methylaminocarbonylaminomethyl)-phenyl]methyl]argininamide;

(R)-N²-(diphenylacetyl)-N-[[4-(ethylaminocarbonylaminomethyl)-phenyl]methyl]argininamide;

(R)-N-[[4-(aminocarbonylaminomethyl)phenyl]methyl]-N²-[bis-(4methoxyphenyl)acetyl]-argininamide;

(R)-N²-(diphenylacetyl)-N-[[4-(ethoxycarbonylmethylaminocarbonylaminomethyl)phenyl]methyl]-argininamide;

(R)-N-[[4-(aminocarbonylaminomethyl)phenyl]methyl]-N²-[bis-(4fluorophenyl)acetyl]-argininamide;

(R)-N-[[4-(aminocarbonylaminomethyl)phenyl]methyl]-N²-[bis-(4chlorophenyl)acetyl]-argininamide;

(R)-N-[[4-(aminocarbonylaminomethyl)phenyl]methyl]-N²-[bis-(4hydroxyphenyl)acetyl]-argininamide;

(R)-N-[[4-(aminocarbonylaminomethyl)phenylmethyl]-N²-[bis-[4(methoxycarbonylmethoxy)phenyl]acetyl]-argininamide; and,

(R)-N-[[4-(aminocarbonylaminomethyl)phenyl]methyl]-N²-[bis-[4(hydroxycarbonylmethoxy)phenyl]acetyl]-argininamide,

and pharmaceutically acceptable salts and hydrates thereof.

Other useful examples of selective NPY Y1 receptor antagonists are disclosed in U.S. Pat. No. 5,962,530 and 6,040,289, which are incorporated herein by reference.

In another embodiment of the invention, the NPY receptor antagonist is selective for the NPY Y5-receptor subtype. An example of a Y5-selective antagonist useful in the present invention is (2-(3,3-dimethyl-1-oxo-4H-1H-xanthen-9-yl)-5,5-dimethylcyclohexane-1,3-dione), known as L-152,804. L-152,804 may be obtained as described by Kanatani et al. in Biochem. Biophys. Res. Commun., 2000, 272:169-173, which is herein incorporated by reference.

When the terms NPY receptor antagonist or NPY antagonist are used herein, it is to be understood that any of the pharmaceutically suitable salts thereof which have NPY receptor antagonist properties in humans and other mammals are included by the term. Such salts include salts with inorganic or organic acids, such as acetic acid, formic acid, hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the NPY antagonist contains a carboxy group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine and triethanolamine.

Pharmaceutical compositions comprising an NPY antagonist and a pharmaceutically acceptable carrier or excipient are effective agents in the therapeutic treatment of alcoholism, thus providing a further aspect of the present invention. Another embodiment of the present invention involves pharmaceutical compositions comprising an NPY Y1selective or an NPY Y5-selective antagonist. Pharmaceutical compositions comprising selective an NPY Y1 antagonist are preferred. Preferred compositions for systemic administration comprise NPY Y1 or Y5 antagonists that cross the blood-brain barrier as administered or in a physiologically activated form.

In the practice of the present invention, the NPY antagonist may be administered systemically or locally provided that the antagonist is available at the site of interaction of NPY with its receptor(s). Preferably the antagonist is administered systemically, for example, parenterally, orally or intraperitoneally. Topical application and aerosol inhalation are also contemplated.

Dosage levels of the order of from about 0.001 mg to about 100 mg of NPY antagonist per kilogram body weight per day are useful in the treatment of alcoholism. The amount of active antagonist that may be combined with carrier materials to produce a single dosage form will vary depending upon the host treated and the particular mode of administration. Dosage unit forms will generally contain between about 1 mg to about 500 mg of an active ingredient.

The specific dose level for any particular individual will depend upon a variety of factors including the activity of the NPY antagonist, the age, body weight, general physical and mental health, genetic factors, environmental influences, sex, diet, time of administration, route of administration, rate of excretion, and the severity of the particular problem being treated. For example, the dose level useful for treating symptoms of alcoholism may vary among individuals depending on the severity of their alcohol abuse problem. Similarly, the dose level for suppressing the craving for alcohol may vary among individuals, depending upon the severity of the individual's alcoholism symptoms. The appropriate dosage within the parameters described herein can be readily determined by one of ordinary skill in the art by routine experimentation using procedures well known in the field.

While it is possible for an active ingredient to be administered alone, it is preferable to present it as a formulation. Formulations of the present invention suitable for oral administration may be in the form of discrete units such as capsules, cachets, tablets, or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. The active ingredient may also be in the form of a bolus, electuary, or paste.

A tablet may be made by compressing or molding the active ingredient optionally with one or more accessory ingredients. Compressed tablets may be prepared by compressing, in a suitable machine, the active ingredient in a free-flowing form such as a powder or granules, optionally mixed with a binder, lubricant, inert diluent, surface active, or dispensing agent. Molded tablets may be made by molding, in a suitable machine, a mixture of the powdered active ingredient and a suitable carrier moistened with an inert liquid diluent.

Formulations suitable for parenteral administration conveniently comprise a sterile aqueous preparation of the active ingredient that is preferably isotonic with the blood of the recipient.

Formulations suitable for nasal or buccal administration, (such as self-propelling powder dispensing formulations described hereinafter), may comprise 0.1 to 20% w/w, for example 2% w/w of active ingredient.

The formulations, for human medical use, of the present invention comprise an active ingredient in association with a pharmaceutically acceptable carrier therefor and optionally other therapeutic ingredient(s). The carrier(s) must be “acceptable” in the sense of being compatible with the other ingredients of the formulations and not deleterious to the recipient thereof.

The pharmacologically active compounds of the invention are useful in the manufacture of pharmaceutical compositions comprising an effective amount thereof in conjunction or admixture with the excipients or carriers suitable for either enteral or parenteral application. Preferred are tablets and gelatin capsules comprising the active ingredient together with one or more of the following: (a) diluents, such as lactose, dextrose, sucrose, mannitol, sorbitol, cellulose, glycine and the like; (b) lubricants, such as silica, talcum, stearic acid, its magnesium or calcium salt, polyethyleneglycol and the like; for tablets also; (c) binders, such as magnesium aluminum silicate, starch paste, gelatin, tragacanth, methylcellulose, sodium carboxymethyl-cellulose or polyvinylpyrrolidone and the like; and, if desired, (d) disintegrants, such as effervescent mixtures and the like; and (e) absorbents, colorants, flavors, and sweeteners and the like. Injectable compositions are preferably aqueous isotonic solutions or suspensions, and suppositories are advantageously prepared from fatty emulsions, or suspensions. Said pharmaceutical compositions may be sterilized and/or contain adjuvants, such as preserving, stabilizing, wetting or emulsifying agents, solution promoters, salts for regulating the osmotic pressure, and/or buffers. In addition, they may also contain other therapeutically valuable substances. Said compositions are prepared according to conventional mixing, granulating, or coating methods, respectively, and contain about 0.1 to 75%, preferably about 1 to 50%, of the active ingredient.

The formulations may conveniently be presented in unit dosage form and may be prepared by any of the methods well known in the art of pharmacy. All methods include the step of bringing the active ingredient into association with the carrier that constitutes one or more accessory ingredients. In general, the formulations are prepared by uniformly and intimately bringing the active ingredient into association with a liquid carrier or a finely divided solid carrier or both, and then, if necessary, shaping the product into the desired formulation.

The effectiveness of the NPY antagonist for its intended use may be determined in a variety of ways. For example, compounds useful in the method of the invention may be selected for further testing on the basis of data from in vitro and/or in vivo animal models. For example, a compound can be evaluated for its binding affinity at the NPY receptor via in vitro bioassays known to those skilled in the art. For example, the method of Kanatani et al., as described in Biochem. Biophys. Res. Commun., 2000, 272:169-173, uses various mammalian cells that are known to express NPY receptor subtypes Y1, Y2, Y4 and Y5 individually. A test compound is considered to be a selective antagonist at a specific receptor subtype if its binding affinity (Kᵢ) is in the nanomolar range in a competitive binding assay against radiolabelled NPY. Another method of determining if a compound is an NPY antagonist is to measure the ability of a test compound to inhibit NPY-induced increases in intracellular Ca²⁺ concentration. This assay is conducted using the same cells as described above in the presence or absence of a test compound and in the presence or absence of NPY. Test compounds that inhibit the ability of NPY to increase intracellular Ca²⁺ with IC₅₀s in the nanomolar range are considered to be antagonists. If a test compound is an antagonist at only one subtype of the NPY receptor, then the compound is considered to be a selective antagonist. A non-selective NPY receptor antagonist shows appropriate binding affinity at two or more receptor subtypes.

The effectiveness of known NPY receptor antagonists and NPY Y1-selective or Y5selective receptor antagonists in reducing craving for alcohol and reducing self-administration of alcohol can be tested in experimental animal models. Briefly, Long-Evans rats are conditioned to the laboratory environment and trained to self-administer concurrent ethanol (10% v/v) vs. water using a sucrose-fading procedure (H. H. Samson, Alcohol Clin. Exp. Res., 1986, 10:436442) as described by Hodge et al. (Alcohol, 1993, 10:191-196). Stereotaxic surgery is performed to implant injector guide cannulae aimed at the PVN. Microinjections of test compounds are given, with and without concomitant injection of NPY, into the PVN of the conditioned rats, which are then immediately given the opportunity to self-administer either water or ethanol (10% v/v). The volume of ethanol and water consumed are measured. Ethanol intake is converted from milliliters consumed to gram/kilogram body weight. Relative ethanol intake (preference) is calculated as milligrams of ethanol consumed divided by total fluid intake (ethanol+water milligrams). Drug dose effects are analyzed by one-way repeated measures analysis of variance (ANOVA). Post-hoc comparisons between drug doses and vehicle control are conducted using a paired t-test or Dunnett's t-test where appropriate (SigmaStat, Jandel, San Rafael, Calif.). A test compound is considered effective when it produces a statistically significant reduction in NPY-induced increases in alcohol self-administration or in baseline self-administration.

Another animal model suitable for testing the effectiveness of the NPY antagonist uses alcohol-reinforced lever pressing behavior as described in Olive et al. Eur. J. Neurosci. 2000 Vol 12, 4131-4140. This model is particularly useful for identifying NPY antagonists that prevent or delay alcohol seeking behavior or prevent or reduce the occurence of relapse drinking in recovering alcoholics.

The general toxicology profile in commonly accepted animal models and bioavailability by the desired route of administration are also considered in the selection of NPY antagonists suitable for use in treatment of alcoholism.

The efficacy of the methods and compositions of the present invention in the treatment of alcoholism, can also optionally be evaluated using procedures that are standard in human clinical trials conducted under appropriate standards and ethical guidelines. For example, a double-blind, placebo-controlled study may be conducted as described by Volpicelli et al., in Arch. Gen. Psychiatry, 1992, 49:876-880. Briefly, subjects who meet the DSM-IV diagnostic criteria for alcohol dependence, including physical signs of alcohol withdrawal, are divided into four treatment groups after receiving standard detoxification therapy: (1) receive test compound; (2) receive placebo compound; (3) receive test compound and behavioral therapy; and (4) receive placebo compound and behavioral therapy. Over a three-month period, all subjects are evaluated on a weekly basis by a research technician who administers a breathalyzer test and obtains measures of craving, alcohol-seeking, alcohol consumption, and moods. The data obtained are analyzed by standard statistical techniques. A test compound is considered effective when it produces a statistically significant reduction in alcohol self-administration, alcohol-seeking behavior, or relapse drinking. For any particular patient, the efficacy of the method can be determined in similar fashion.

The following examples are offered by way of illustration and not by way of limitation.

### EXAMPLES

Methods

Animals. Thirty-six male Long-Evans rats (Harlan, Indianapolis, Ind.) were housed individually in Plexiglas cages. Access to water was restricted during the first day of behavioral training, but was continuously available for the remainder of the experiment. Food was always available in the home cage. In experimental chambers, ethanol (10% v/v) and water were available concurrently during daily (M-F) sessions. The animal colony room was maintained on a 12L:12D cycle with the lights on at 06:30. Experimental sessions were run during the light portion of the cycle. All experimental procedures were conducted under institutional and NIH guidelines.

Apparatus. Experimental sessions were conducted in Plexiglas chambers (27×37×21 cm) located in sound-attenuating cubicles (MED Associates, model ENV 016M, Lafayette, Ind.). Chambers were equipped with exhaust fans that masked external noise. The right wall of each chamber contained two 50-ml drinking bottles. Drug solutions were administered bilaterally through stainless steel injectors (Plastics One, Roanoke, Va.), which were connected via plastic tubing to two 1.0 μl syringes (Hamilton, Reno, Nev.). Syringes were mounted on a micro-infusion pump (Harvard Apparatus, Model 22, Natick, Mass.) set to deliver 0.5 μl/min/syringes.

Self-administration procedures. After 2 weeks of adaptation to laboratory housing conditions, fluid access was restricted to 1-h per day (for 2 days only) and two groups of rats (n=12 per group) were trained to drink from the bottles in the experimental chambers by overnight access to sucrose (10% w/v) vs. water during one overnight session. Daily 1 hour sessions were then conducted with sucrose (10% w/v) vs. water available concurrently. The location (i.e., left or right side of the chamber) of the sucrose and water solutions was alternated daily. When sucrose and water intake patterns stabilized, the rats were trained to self-administer concurrent ethanol (10% v/v) vs. water using a sucrose-fading procedure (H. H. Samson, Alcohol Clin. Exp. Res., 1986, 10:436-442) as previously described (e.g., Hodge et al., 1993). Briefly, ethanol was gradually added to the sucrose solution and then sucrose was faded out of the solution until rats were self-administering 10% ethanol vs. water. During the 2-month sucrose-fading procedure, the location of the ethanol/sucrose solution and water were alternated daily. All animals preferred the ethanol/sucrose solution to water (data not shown). After sucrose fading, the rats were allowed to self-administer ethanol (10% v/v) versus water 5 days per week (M-F) for 4 months to establish a long-term history of ethanol self-administration. At the end of the 4-month baseline procedure, all animals underwent stereotaxic surgery. They were then allowed to self-administer ethanol for an additional 3 months prior to microinjection procedures.

Since NPY has potent effects on consummatory behavior, it is possible that NPY infusions in the PVN could alter ethanol intake via mechanisms that are unrelated to experience with the pharmacological effects of ethanol. Thus, as a control for nonspecific effects of NPY on consumption, the effects of NPY were tested in a separate group of rats (n=12) that did not have a long-term history of ethanol self-administration. Experimentally naive rats were allowed one week to adapt to the laboratory. They were then implanted with bilateral guide cannulae aimed at the PVN. Experimental sessions began one week after surgery to allow for recovery. During daily 60-min sessions, rats were placed in the self-administration chambers with concurrent ethanol (10% v/v) vs. water available. Baseline levels of ethanol and water intake were measured for one week. Microinjection procedures were then conducted.

Stereotaxic surgery. When ethanol and water intake stabilized, bilateral stainless steel guide cannulae (26 gauge) aimed at the PVN were surgically implanted. Rats were anesthetized with pentobarbital (60 mg/kg, i.p.) combined with atropine (0.4 mg/kg, i.p.) and placed in a stereotaxic instrument (David Kopf Instruments, Tujunga, Calif.). Injector cannulae (Plastics One, Roanoke, Va.), aimed to terminate 1 mm dorsal to the PVN, were implanted and secured to the skull with cranial screws and dental cement. Removable wire obturators were inserted in the full length of the guide cannulae to limit obstruction by tissue and contamination by external debris. The stereotaxic coordinates used for the PVN were −1.8 mm from bregma, +1.0 mm lateral to the midline, and −6.5 mm ventral to the cortical surface at 5° lateral to the vertical plane (Paxinos & Watson, 1982). All measurements were taken from flat skull. Following surgery, all rats were given buprenorphine (0.2 mg/kg, sc.) for post-operative pain management. Daily sessions were resumed one week after surgery.

Microinjection procedure. When ethanol and water intake stabilized again, microinjections were conducted once per week. Unanesthetized rats were placed in plastic containers (15×30×15 cm deep) to reduce movement. Obturators were removed and sterile 33-gauge injectors were inserted bilaterally to a depth 1 mm beyond the end of the guide cannulae. Drug solutions were infused bilaterally in distilled water vehicle in a total volume of 1 μl (0.5 μl/side) over a 1-min period. The injectors were left in place for an additional 30-sec period to allow drug diffusion. Precise flow of the solutions was verified before and after each injection to ensure compound delivery. Self-administration sessions began immediately after microinjections. Sterile obturators were reinserted at the end of the behavioral sessions. When co-administered, D-NPY or BIBP 3226 was infused 15-min prior to NPY. Vehicle injections were also performed to control for local pressure or osmotic changes caused by infusions. During one month prior to drug testing, the animals were handled and placed in the plastic tubs to minimize the effects of procedural changes on subsequent drug effects. The data from these sessions were not used in the analysis. After completion of the microinjection protocol, the rats were sacrificed and their brains were removed for histological verification of injection sites.

Drugs and dosing. The drugs used in this study for central administration were NPY, the non-selective NPY antagonist [D-Tyr^{27,36}D-Thr³²] Neuropeptide Y (27-36) (DNPY), and the Y1-selective antagonist (R)-N²-(Diphenylacetyl)-N-[(4hydroxyphenyl)methyl]-D-arginine amide (BIBP 3226). All drugs were obtained from Research Biochemicals International, Natick, Mass. All drugs were dissolved in sterile distilled water for central administration. Drug solutions were prepared immediately prior to administration and were infused bilaterally in a total volume of 1 μl (0.5 μl/side/min). Drug doses were administered in a randomized order by an experimenter not blinded to dose. Ethanol (95%) was diluted in tap water for self-administration.

Histology. After completion of the experiment, the rats were administered a lethal dose of sodium pentobarbital (200 mg/kg, ip.) and perfused transcardially with 0.9% NaCl followed by 10% formalin. The brains were removed and stored in a solution of 10% formalin/30% sucrose for a minimum of 10 days. Fixed brains were frozen, sectioned (40 μm), and stained with cresyl violet and examined under a light microscope to determine injection locations. Data were used only from rats that were verified to have clearly definable injector tracks that terminated bilaterally in the PVN.

Data analysis and statistics. Volume of ethanol and water consumed was measured to the nearest 0.5 milliliter at the end of each 1-h session. Ethanol intake was converted from mls consumed to g/kg body weight. Relative ethanol intake (preference) was calculated as mls of ethanol consumed divided by total fluid intake (ethanol+water mils). Drug dose effects were analyzed by one-way repeated measures analysis of variance (ANOVA). Post-hoc comparisons between drug doses and vehicle control were conducted using paired t-test or Dunnett's t-test where appropriate (SigmaStat, Jandel, San Rafael, Calif.).

Results

The behavioral procedures resulted in stable ethanol self-administration in the majority of animals. Ethanol intake averaged over the 25 days preceding microinjection procedures was (0.44±0.05 g/kg). Data from 3 rats in the group that received NPY and BIBP-3226 were excluded from analysis due to deterioration of baseline performance.

Histology. Analysis of histological examination of coronal brain sections showed that 11 of 12 animals that received sucrose fading followed by NPY and D-NPY infusions received bilateral injections in the PVN. Examination of brain sections from rats that received sucrose fading followed by NPY and BIBP 3226 showed that 9 of 9 animals received injections in the PVN. Ten of 12 control animals received bilateral injections in the PVN. The injection procedure produced minimal gliosis or tissue damage in the PVN. Data are presented only for animals that received bilateral injections in the PVN.

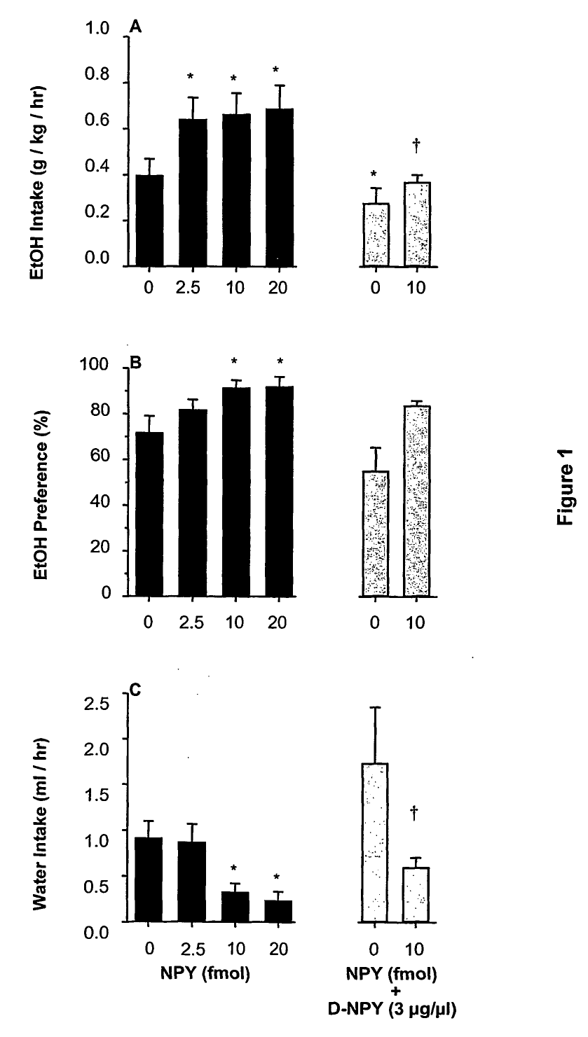

Effects of NPY on ethanol intake and preference. The results of NPY infusions in the PVN are shown in FIG. 1. Repeated-measures ANOVA indicated that NPY significantly increased ethanol intake [F(3,30)=7.6, p<0.001]. Post-hoc Dunnett's comparisons showed that all doses of NPY significantly increased ethanol intake, as compared to vehicle control (FIG. 1A). Inspection of data from individual rats indicated that all eleven rats tested showed increased ethanol intake (g/kg) at all doses of NPY. Although baseline ethanol preference was high (>70%), NPY administration in the PVN also significantly increased ethanol preference [F(3,30)=3.3,p<0.05] in a dose-dependent manner (FIG. 1B). At the two highest doses of NPY tested, ethanol represented greater than 90% of fluid consumption during daily sessions (p<0.05; FIG. 1B). The increases in ethanol intake and preference were associated with a significant overall reduction in water intake [F(3,30)=5.4,p<0.01] during experimental sessions (FIG. 1C). Post-hoc comparisons demonstrated that NPY decreased water intake in a dose-dependent manner (p<0.05).

The nonselective NPY receptor antagonist D-NPY produced a partial, but significant, decrease in ethanol intake (FIG. 1A, right). When co-administered with NPY (10 fmol), D-NPY completely blocked the effects of NPY on ethanol and water intake (FIGS. 1A and 1C, right). Although there was a trend in the preference data, D-NPY did not significantly alter NPY-induced increase in ethanol preference, P<0.09 (FIG. 1B). Therefore, these data show that infusion of exogenous NPY in the PVN potently increased ethanol intake and preference in rats. The nonselective NPY antagonist D-NPY partially reduced baseline ethanol intake and completely blocked the increase produced by NPY.

Table 1 shows the effects of NPY infusion in the PVN of rats that had no long-term history of ethanol self-administration that underwent the sucrose-fading procedure. Data values are presented as mean±SEM. Under these conditions, no dose of NPY tested produced a change in ethanol intake or preference, or water intake.

TABLE 1

| | NPY (fmol) | | |
|---|---|---|---|
| | 0.0 | 10.0 | 100.0 |
| ETOH | 0.071 ± 0.026 | 0.0330 ± 0.024 | 0.0550 ± 0.03 |
| Intake (g/kg/hr) | | | |
| ETOH | 43.33 ± 15.755 | 20.0 ± 13.333 | 30 ± 15.275 |
| Preference (%) | | | |
| Water | 0.1667 ± 0.118 | 0 ± 0 | 0.3 ± 0.133 |
| Intake (ml/hr) | | | |

Comparable effects of carbohydrate and alcohol intake have been known for some time (C. P. Richter, Q. J. Stud. Alcohol, 1953 14:525-539; P. J. Kulkosky, Neurosci. Biobehav. Rev., 19859:179-190). Alcohol intake decreases carbohydrate intake in a nutrient-selective manner without altering protein or fat consumption of rats (O. A. Forsander, Alcohol, 1988, 23:143-149). Accordingly, human studies indicate that intake of sweets is inversely related to alcohol consumption (G. A. Colditz et al., Am. J. Clin. Nutr., 1991, 54:49-55). Other studies have demonstrated that low carbohydrate diets increase alcohol intake, but high carbohydrate diets decrease alcohol drinking (R. V. Brown et al., Q. J. Stud. Alcohol, 1973, 34:758-763; L. Pekkanen et al., Br. J. Nutr., 1978, 40:103-113; O. A. Forsander et al., Alcohol, 1988, 5:233-238). Simple carbohydrates have been reported to help delay relapse in alcoholics (M. E. Farcas et al., J. Nutr. Education, 1984, 16:123-124). Furthermore, recovering alcoholic women have been noted to eat more sweet foods during periods of strong alcohol craving (S. Rosenfield, Res. Nurs. Health, 1988, 11:165-174). Therefore pairing ethanol with a carbohydrate, such as sucrose, during the development of self-administration behavior may engage homeostatic neural systems that mediate food intake (i.e., the hypothalamus). Alternatively, it is possible that the higher level of ethanol intake by sucrose-trained animals influenced the effect of NPY.

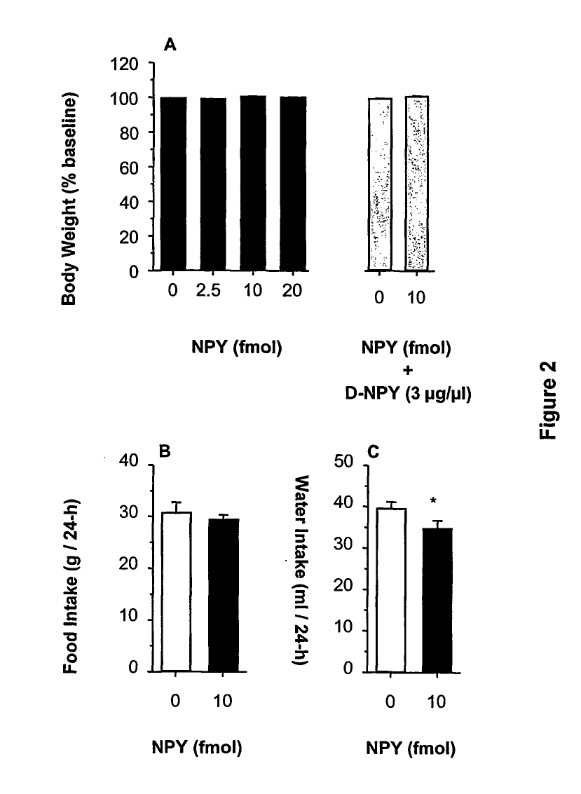

Effects of NPY on home-cage food and water consumption. NPY infusion in the PVN had no effect on body weight (FIG. 2A) or food consumption (FIG. 2B) measured in the home cage (FIG. 2B). However, this dose of NPY significantly F(1,10)=9.625,p<0.05] reduced water intake in the home cage (FIG. 2C). Administration of D-NPY either alone or in combination with NPY produced no effect on body weight (FIG. 2A, right). NPY also produced no effect on body weight, food intake, or water intake in the home cage of sucrose-inexperienced animals.

The absence of NPY-related effects on food intake and body weight might be accounted for by several factors. First, the concentrations of NPY used in the present study were in the fmol range, which is significantly less than concentrations typically used to induce feeding, which are in the pmol-nmol range (e.g., Stanley and Leibowitz, 1985). Thus, ethanol self-administration behavior may be more sensitive to alterations in NPY levels than feeding. Second, elevated NPY levels in the PVN may produce immediate increases in consumption of the most relevant substance in the environment. This did not appear to be the case in the present experiment because NPY produced no effect on ethanol intake in rats trained with a sucrose-independent method.

NPY significantly decreased water intake during ethanol self-administration sessions, which contributed to the increase in ethanol preference. We also observed relatively small but significant reductions in 24-h water intake in the home cage after NPY infusion in the PVN. The non-selective NPY antagonist D-NPY significantly reversed NPY-induced decreases in water intake during ethanol self-administration sessions and the Y1-selective antagonist reversed a trend toward decreased water intake. Taken together, these data suggest that NPY has differential effects on water intake in ethanol-experienced versus ethanol-inexperienced rats.

Effects of Y1 Antagonist on NPY-Induced Changes in Ethanol Intake and Preference.

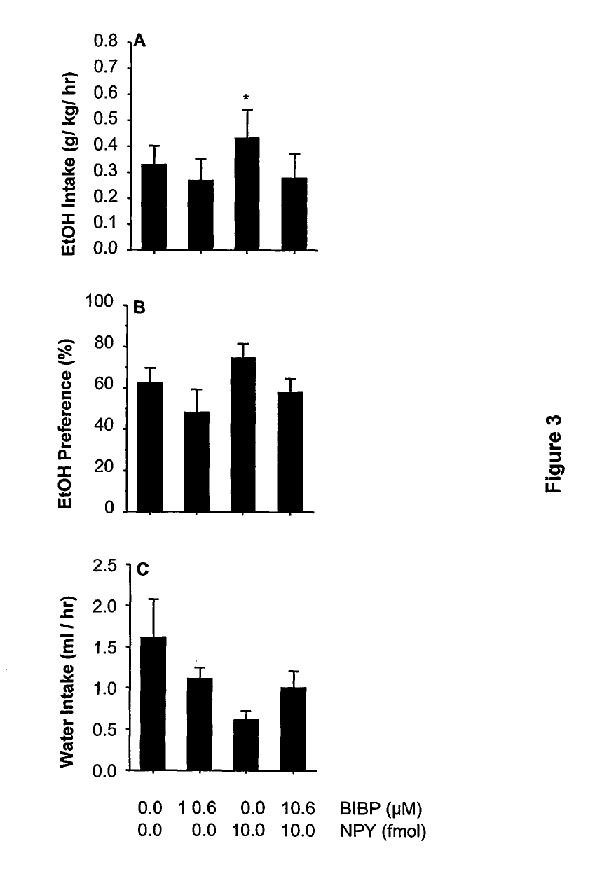

FIG. 3 shows the results of intra-PVN infusion of NPY and the NPY Y1-selective antagonist BIBP 3226. Infusion of BIBP 3226 (10.6 μm) alone in the PVN produced no significant effect on ethanol intake or preference, or water intake (FIGS. 3A, B, and C). NPY (10 fmol) significantly increased ethanol intake above control values (FIG. 3A). Co-administration of BIBP 3226 with NPY in the PVN completely blocked the ability of this dose of NPY to increase ethanol intake (FIG. 3A). Therefore, these data indicate that NPY, acting at Y1 receptors in the PVN, is a potent stimulant of alcohol self-administration and that a specific Y1 receptor antagonist can completely block this stimulation.

Microinjection of NPY-Y1 Antagonist in the Amygdala

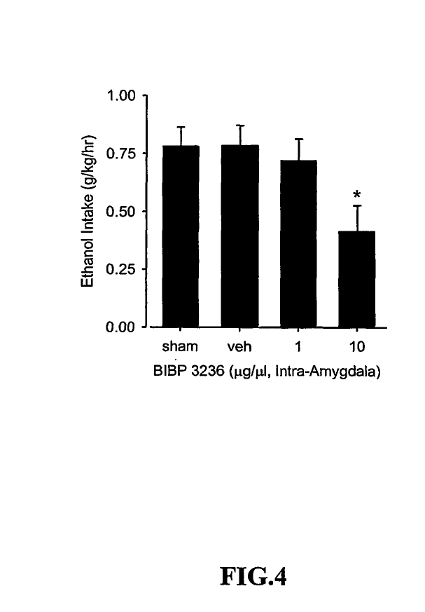

To further elucidate the role of NPY receptors in alcohol-seeking behavior, the NPY-Y1 peptide-antagonist BIBP 3226 was injected in the central nucleus of the amygdala (CeA) of rats trained to self-administer ethanol vs. water as described (Kelley et al., 2001 Peptides 22: 515-522). The CeA was chosen as an additional test site because, in addition to the hypothalamus, this brain region contains significant numbers of NPY receptors. The antagonist produced no effect on water intake. Repeated measures ANOVA showed that BIBP 3226 but significantly altered the dose of self-administered ethanol [F(3, 23=5.9, p<0.01]. Follow up statistical analysis indicated that the significant main effect was due to reduced ethanol self-administration after infusion of the highest dose of BIBP 3226 (FIG. 4). These data indicate that blockade of NPY-Y1 receptors in the CeA reduces the reinforcing efficacy of ethanol. This reduction in ongoing ethanol self-administration suggests that Y1 antagonists are useful therapeutic agents in the medical management of problems associated with alcohol abuse and alcoholism, such as uncontrolled drinking.

Systemic Injection of the Non-Peptide NPY-Y5 Antagonist L 152,804

Evidence from our laboratory indicates that microinjection of NPY increases alcohol self-administration. This effect was blocked by infusion of either a non-selective NPY antagonist (D-NPY) or BIBP 3226 in the hypothalamus. Evidence shown above extended these findings by demonstrating that the Y1 antagonist BIBP 3226 administered in the CeA significantly reduced alcohol self-administration. These data demonstrate potential efficacy of NPY antagonists as therapeutic agents in the medical management of alcohol abuse and alcoholism. However, the generality of these findings is somewhat limited by the fact that the compounds were administered directly into specific brain regions. To extend the relevance of these findings, we synthesized and tested a recently reported (Kanatani et al., 2000) selective non-peptide NPY-Y5 antagonist, L152,804, on alcohol-seeking behavior. As a stringent test of compound efficacy, we tested the Y5 antagonist in C57BL/6J mice, which have a genetic pre-disposition to self-administer high doses of alcohol.

Methods

Male C57BL/6J mice (N=8) were housed in standard Plexiglas cages (n=4/cage) with food (Harlan, Indianapolis, Ind.) and water always available. Mice were 20 weeks of age (body weight, 28-35 g) and drug naïve at the start of all testing. During operant self-administration training, food pellets (15 g per overnight session) were placed into the operant chambers and water was available under a fixed ratio-1 (FR-1) schedule of reinforcement.

Test sessions were conducted in 8 Plexiglas operant chambers (Med Associates, Lafayette, Ind.) measuring 15.9×14×12.7 cm with stainless steel grid floors. Each chamber was housed in a sound-attenuating cubicle equipped with a house fan that provided ventilation and helped mask external noise. The left and right wall of each operant chamber was equipped with one ultra-sensitive stainless steel response lever and a liquid delivery system. Liquid solutions (ethanol or water) were maintained in 60 ml syringes mounted on a programmable pump (PH-100, Med Associates), which delivered 0.01 ml per activation into a stainless steel cup located to the left of the associated response lever. Each chamber also contained a house light (illuminated between 16:00-18:00 hr and 06:00-08:00 hr), as well as a stimulus light located above each lever (activated each time the lever was pressed). The chambers were interfaced (Med Associates) to an IBM-compatible PC, which was programmed to record all lever presses and liquid deliveries.

Mice were trained to lever press using reinforcement (10% sucrose w/v) of successive approximations. After initial behavioral shaping sessions, mice were run during 16 hr overnight (16:00-08:00 hrs) training sessions. During these training sessions, both response levers were active on a concurrent fixed ratio one (CONC FR1 FR1) schedule with 10% sucrose vs. water presented as the reinforcer. The position of each solution (left or right) was fixed for each animal but counterbalanced between animals to control for side preference. After 4 days, mice were trained to orally self-administer ethanol (10% v/v) vs. water using a sucrose substitution procedure, which we have adapted for use in the mouse (Olive et al., 2000 Eur. J. Neurosci. 12:4131-4140). Briefly, ethanol (2, 4, 8, or 10% v/v) was incrementally added to the sucrose (10% w/v) solution with 4 days at each increasing concentration. Then, sucrose (10, 5, 2% w/v) was incrementally faded out of the ethanol containing solution with 4 days at each decreasing concentration. After sucrose substitution training, all mice reliably responded on the CONC FR1 FR1 schedule of reinforcement with ethanol (10% v/v) vs. water presented as the reinforcers.

Effects of L 152,804 of Alcohol-Seeking Behavior

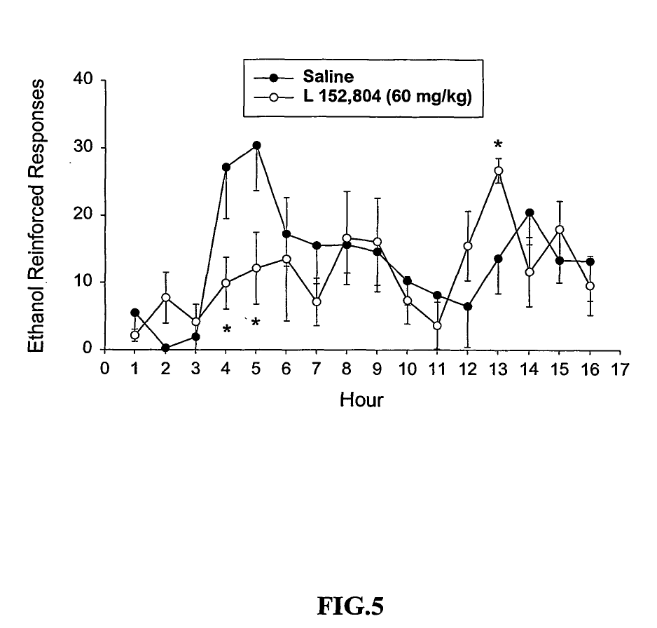

Systemic administration of L 152,804 (10, 30, and 60 mg/kg) produced no effect on water intake during 16-h sessions. Analysis of control alcohol self-administration performance showed that behavior occurred throughout the session but peaked after 4-5 hours of access (FIG. 5, closed circles). Administration of the two low doses of L 152,804 did not significantly alter ethanol self-administration. However, L 152,804 (60 mg/kg) significantly decreased alcohol-seeking behavior (e.g., lever presses for alcohol) during the peak period (FIG. 5, open circles). A compensatory increase in self-administration occurred during the 13^{th} hour of access, which might correspond with the pharmacokinetics of the Y5 antagonist. These data suggest that L 152,804 reduced alcohol-seeking behavior during a period of high motivation.

Effects of L 152,804 on the Onset of Alcohol-Seeking Behavior

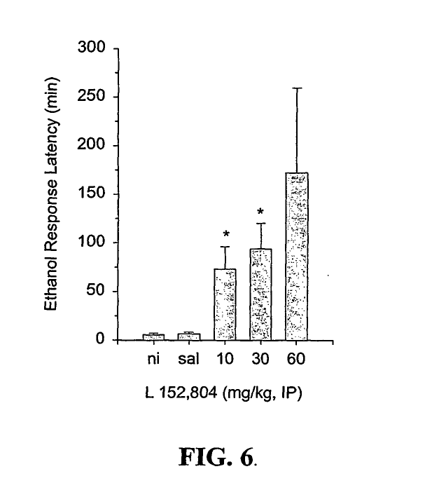

Each daily self-administration session represents a cue-induced (i.e., experimental environment) opportunity to either seek ethanol or not, much like cue-induced relapse procedures that deprive animals of access to drugs for some period of time. In the present study, the deprivation period was from 8:00 to 16:00 hrs each day. Thus, to further address the potential effects of L 152,804 on motivation to self-administer ethanol (and relapse), we analyzed the time at which responding began during each session. L 152,804 significantly increased the latency to the first response (FIG. 6). These data indicate that the NPY Y5 antagonist delayed the onset of ethanol seeking behavior. By extension of the animal model to the human condition of alcoholism, these data suggest that NPY Y5 antagonists might help delay relapse in alcoholics attempting to avoid seeking the drug.

All publications and patent applications mentioned in this specification are herein incorporated by reference to the same extent as if each individual publication or patent application was specifically and individually indicated to be incorporated by reference. Unless defined otherwise, all technical and scientific terms used herein have the same meaning as commonly understood by one of ordinary skill in the art to which this invention belongs.

The invention now being fully described, it will be apparent to one of ordinary skill in the art that many changes and modifications can be made thereto without departing from the spirit or scope of the appended claims.

What is claimed is:

1. A method for reducing self-administration of alcohol in a patient suffering from alcoholism comprising administering to said patient a therapeutically effective amount of an NPY receptor antagonist and determining the level of alcohol self-administration in said patient before and after said administering.

2. The method according to claim 1, wherein said NPY receptor antagonist is a selective NPY Y1 receptor antagonist.

3. The method according to claim 2, wherein said selective NPY Y1 receptor antagonist is BIBP 3226.

4. The method according to claim 1, wherein said NPY receptor antagonist is a selective NPY Y5 receptor antagonist.

5. The method according to claim 4, wherein said NPY receptor antagonist is 2-(3,3-dimethyl-1-oxo-4H-1H-xanthen-9-yl)-5,5-dimethyl-cyclohexane-1,3-dione or a pharmaceutically acceptable salt or hydrate thereof.

6. A method for reducing alcohol-seeking behavior in a patient suffering from alcoholism comprising administering to said patient a therapeutically effective amount of an NPY receptor antagonist and determining the level of alcohol-seeking behavior in said patient before and after said administering.

7. The method according to claim 6, wherein said NPY receptor antagonist is a selective NPY Y1 receptor antagonist.

8. The method according to claim 7, wherein said selective NPY Y1 receptor antagonist is BIBP 3226.

9. The method according to claim 6, wherein said NPY receptor antagonist is a selective NPY Y5 receptor antagonist.

10. The method according to claim 9, wherein said NPY receptor antagonist is 2-(3,3-dimethyl-1-oxo-4H-1H-xanthen-9-yl)-5,5-dimethyl-cyclohexane-1,3-dione or a pharmaceutically acceptable salt or hydrate thereof.

11. A method for preventing or reducing the occurrence of relapse drinking in a recovering alcoholic patient comprising administering to said patient a therapeutically effective amount of an NPY receptor antagonist and determining the frequency of occurrence of relapse drinking in said patient before and after said administering.

12. The method according to claim 11, wherein said NPY receptor antagonist is a selective NPY Y1 receptor antagonist.

13. The method according to claim 12, wherein said selective NPY Y1 receptor antagonist is BIBP 3226.

14. The method according to claim 11, wherein said NPY receptor antagonist is a selective NPY Y5 receptor antagonist.

15. The method according to claim 14, wherein said NPY receptor antagonist is 2-(3,3-dimethyl-1-oxo-4H-1H-xanthen-9-yl)-5,5-dimethyl-cyclohexane-1,3-dione or a pharmaceutically acceptable salt or hydrate thereof.

16. A pharmaceutical composition for the treatment of alcoholism in a mammal afflicted therewith comprising a therapeutically effective amount of an NPY receptor antagonist.
